(19) United States
(12) Patent Application Publication
(10) Pub. No.: US 2024/0349755 A1
(43) Pub. Date: Oct. 24, 2024

(54) EDIBLE PLANT-BASED PROTEIN COMPOSITION

(71) Applicant: MEALA FOODTECH LTD, Haifa (IL)

(72) Inventors: Liran GARUDA, Haifa (IL); Hadar EKHOIZ RAZMOVICH, Givat Shapira (IL); Tali FELDMAN SIVAN, Hadar Am (IL); Vered BEN YOSEF SHUSTER, Bustan HaGalil (IL)

(21) Appl. No.: 18/685,184

(22) PCT Filed: Aug. 28, 2022
(86) PCT No.: PCT/IL2022/050932

§ 371 Date: Feb. 20, 2024

Related U.S. Application Data
(60) Provisional application No. 63/238,172, filed on Aug. 29, 2021.

(51) Int. Cl. A23J 3/22; A23L 29/00; A23L 33/18; C12N 9/02; C12N 9/10; C12N 9/18; C12N 9/26; C12N 9/42

(52) U.S. Cl. CPC A23J 3/227; A23L 29/06; A23L 33/18; C12N 9/0071; C12N 9/1044; C12N 9/18; C12N 9/2414; C12N 9/2437; C12Y 114/18001; C12Y 301/01011; C12Y 302/01015

(57) ABSTRACT

A system and method for producing a composition comprising plant derived polypeptides, and at least one enzyme capable of crosslinking said plant derived polypeptides. The composition comprising a porous plant protein matrix comprising crosslinked plant derived polypeptides, and at least one enzyme capable of catalyzing amino acid oxidation and/or at least one enzyme capable of forming peptide bonds between amino acid residues, wherein the matrix is capable of forming a hydrogel when hydrated.

### FIELD OF THE INVENTION

Provided herein is a composition comprising plant derived polypeptides, and at least one enzyme capable of crosslinking said plant derived polypeptides.

### BACKGROUND

The plant-based category is growing yearly, and, with it, the demand for plant-based products. Appropriately, the number of people following plant-based diets is increasing tremendously. According to the Blue Horizon, by 2035, every tenth portion of meat, eggs, and dairy food products eaten globally will likely be plant-based.

Many customers believe that plant-based products are natural and healthy, giving rise to the popularity of alternative food products. However, many of the plant-based food products seen on the market are ultra-processed therefore, there is a disconnect between the products currently available to consumers and the concept that plant-based products are healthier than meat-based products.

Existing products do not provide an adequate response to the market's needs, as evident by the bloated and incomprehensible ingredient lists of heavily processed products. To bring about substantial growth in the market through significant reductions in the consumption of animal-based foods, a significant change in the raw materials is required. The optimal solution should involve a clean product label that facilitates the shopping experience while also improving consumer satisfaction via a positive taste and texture experience.

Plant proteins require a high degree of processing and manipulation to mimic the sensory properties of meat. Clark and Bogdan found that products using alternative protein are considered “too processed” and “high in sodium” by the group unlikely to purchase them. Currently, the industry does not create raw materials specifically designed for the plant-based industry.

Plant-based proteins cannot crosslink and have low water retention capacity. Therefore, they require the addition of gelling agents such as methylcellulose or other hydrocolloids to behave similarly to their animal-based counterparts, that is, to imitate the texture of meat, egg, and fish in alternative food products.

Methylcellulose is a cellulose derivative used as a thickener, emulsifier, binder, stabilizer, and gelling agent in food and has the European food additive number E461. It is a water-soluble polymer chemically modified from natural cellulose by partial etherification. Methylcellulose forms a gel that gels upon heating above certain temperatures (generally, 42.5° C.) and returns to become a viscous solution after cooling down.

Therefore, using methylcellulose and other hydrocolloids in alternative food products necessitates using additional food additives, such as stabilizers, taste maskers, etc. to perform their function properly. Therefore, incorporating them into a product result in a bloated list of ingredients, which goes against the move to clean labeling. Furthermore, their usability is far from ideal, and their produced taste and texture do not accurately mimic their meat counterparts.

Additionally, methylcellulose and its derivatives such as carboxymethylcellulose (CMC), while in use since the 1960, have been found to alter the gut microbiome, resulting in disease in the form of various chronic inflammatory conditions, including colitis, metabolic syndrome, and colon cancer.

While there is impressive market growth, the industry still faces several challenges in fulfilling alternative food products' true potential, such as clean label, usability, taste and texture, and price.

Therefore, there is a need to create better plant-based products by developing healthier, clean label ingredients, thereby enabling the production of better products that fulfill the category's promise of healthier food.

### SUMMARY

Some embodiments of the present disclosure provide a combination of enzymes and physical treatment of plant-based proteins to give them meat, fish, dairy or egg protein like properties.

According to some embodiments, a composition is described herein comprising a porous plant protein matrix comprising crosslinked plant derived polypeptides, and at least one enzyme capable of catalyzing amino acid oxidation and/or at least one enzyme capable of forming peptide bonds between amino acid residues, wherein the matrix may be capable of forming a hydrogel when hydrated. Preferably, the composition is devoid of synthetic gelling agents such as but not limited to methyl cellulose.

According to some embodiments, the composition may comprise a plant derived protein. According to some embodiments, the composition may comprise a plant derived polypeptide.

According to some embodiments, the plant protein may be derived from a plant protein isolate, a plant protein concentrate and/or a plant derived flour, collectively referred to as “plant protein source”. Each possibility is a separate embodiment. According to some embodiments, the composition comprises at least 80% w/w, at least 85% w/w, at least 90% or at least 95% plant protein source

As used herein, the term plant “protein isolate”, refers to proteins extracted from plants by various methods such as isoelectric precipitation separation and ultrafiltration to obtain a highly concentrated protein fraction. The protein isolate typically includes at least 80% or at least 90% w/w plant proteins. Each possibility is a separate embodiment.

As used herein, the term plant “protein concentrate”, refers to proteins extracted from plants but without the additional processing steps of reducing fat and carbohydrate content carried out to obtain a protein isolate, the proteins per scoop is therefore lower in a protein isolate. The protein concentrates typically include 40-80% w/w plant proteins, such as about about 40% w/w, about 50% w/w, about 60% w/w, about 70% w/w or about 75 w/w plant proteins. Each possibility is a separate embodiment.

As used herein, the term plant “protein flour” refers to flours obtained from plants with high protein content. Protein rich flour typically include 10-50% or 12-40% w/w plant proteins, such as about 10% w/w, about 12% w/w, about 15% w/w, about 20% w/w, about 25% w/w or plant proteins. Each possibility is a separate embodiment. Non-limiting examples of protein rich flours include chickpea flour (˜22%), coconut flour (˜20%), peanut flour (˜34%), red lentil flour (26%), sesame flour (˜40%), soy flour (˜38%), sunflower seed flour (˜48%), almond flour (˜21%).

According to some embodiments, the matrix may comprise at least about 80% w/w, at least about 85% w/w, at least about 90% w/w, or at least about 95% w/w of the plant protein source when in a powder (dehydrated) form. Each possibility is a separate embodiment. According to some embodiments, the composition may comprise at least about 50% w/w, at least about 60% w/w, at least about 70%, at least about 80% w/w, at least about 85% w/w, at least about 90% w/w, or at least about 95% w/w of the plant protein source, when in a powder (dehydrated) form.

According to some embodiments, the protein source to enzyme ration may be in a range of 1:0.005-1:0.2, or 1:0.01-1:0.1 or 1:0.01-1:0.06 or 1:0.02-1:0.05. Each possibility is a separate embodiment.

According to some embodiments, the plant-based polypeptide may be derived from the group consisting of pea, soy, corn, wheat, rice, beans, seed, nut, almond, peanut, seitan, lentil, chickpea, flaxseed, chia seed, quinoa, oat, buckwheat protein, bulgur, millet, microalgae, hemp, sunflower, canola, lupin, legumes, potato, wild rice, fava bean, yellow pea protein, mung bean, and combinations thereof. Optionally, the composition may be devoid of animal derived proteins and/or fats.

According to some embodiments, the at least one enzyme capable of catalyzing amino acid oxidation may be an oxidoreductase. Optionally, the oxidoreductase may be a multicopper enzyme capable of oxidating phenolic residues. Optionally, the oxidoreductase may be a laccase, a tyrosinase, a peroxidase, a glutathione oxidase or any combination thereof.

According to some embodiments, the at least one enzyme capable of forming the peptide bonds may be a transferase or a peptidase. Optionally, the transferase may be an amino-acyltransferases, preferably a protein-glutamine gamma-glutamyltransferase. Optionally, the peptidase may be a cysteine endopeptidase.

According to some embodiments, the at least one enzyme capable of catalyzing amino acid oxidation and/or the at least one enzyme capable of forming peptide bonds between amino acid residues may be reversibly inactivated by drying and/or freezing. Optionally, the reversibly inactive enzyme may be reactivated upon hydration and/or thawing.

According to some embodiments, the composition may include an enzyme capable of hydrolyzing polysaccharides. Optionally, the enzyme capable of hydrolyzing polysaccharides may be a pectinase, an amylase, a cellulase or any combination thereof.

According to some embodiments, the composition may include a lipase. Optionally, the lipase may be selected from a phospholipase, a lysophospholipase, a galactolipase, a feruloyl esterase or any combination thereof.

According to some embodiments, the plant protein matrix may include a mediator mediating crosslinking of the polypeptides. According to some embodiments, the composition may include one or more co-factors, vitamins, minerals and/or combination thereof.

According to some embodiments, the composition may be in the form of a powder, a solid, a hydrogel and/or a mixture thereof. According to some embodiments, the composition may be in the form a hydrogel. Optionally, the hydrogel may be thermoresistant. Optionally, the hydrogel may comprise active residues which upon rehydration enable crosslinking between the matrix and externally added polypeptides.

According to some embodiments, a food product may include the composition. Optionally, the food product may include between about 1-50% w/w, between about 1-25% w/w, or between about 1-15% w/w of the composition. Each possibility is a separate embodiment.

According to some embodiments, the food product may be a plant-based meat alternative product, plant-based fish alternative product, egg-less egg alternative product, a dairy replacement product, a chocolate alternative product, an egg-less bakery product, a hybrid meat-plant-based meat alternative product, a hybrid fish-plant-based fish alternative product, a hybrid dairy-plant-based dairy alternative product, a hybrid egg-plant-based egg alternative product, or a combination thereof. Each possibility is a separate embodiment.

According to some embodiments, a process for producing a porous plant protein matrix capable of forming a hydrogel when hydrated may include mixing plant-derived polypeptides with at least one enzyme capable of catalyzing amino acid oxidation and/or at least one enzyme capable of forming peptide bonds between amino residues, and incubating at conditions allowing crosslinking of at least a portion of the plant derived polypeptides, thereby forming a hydrogel. According to some embodiments, the concentration of the plant derived protein in the hydrogel may be less than about 30% w/w, less than 20% w/w or less than 10% w/w. Each possibility is a separate embodiment. According to some embodiments, the concentration of the plant derived protein in the hydrogel may be about 5-30% w/w or about 10-30% w/w. According to some embodiments, the hydrogel comprises at least about 60% w/w, at least about 70% w/w, at least about 80% w/w or at least about 90% w/w water.

According to some embodiments, the process may include a step of preprocessing the plant-based polypeptides prior to and/or during the mixing to expose amino acid residues. Optionally, the preprocessing may include heating, pressure, sonication treatment or any combination thereof. Each possibility is a separate embodiment.

According to some embodiments, the process may include a step of generating a semi-activated enzyme mixture. Optionally, the mixture may include the at least one enzyme capable of catalyzing amino acid oxidation and/or the at least one enzyme capable of forming peptide bonds and/or an enzyme capable of degrading polysaccharides.

According to some embodiments, the process may include adding at least one enzyme comprises capable of degrading polysaccharides to the plant-derived polypeptides prior to the mixing with the at least one enzyme capable of catalyzing amino acid oxidation and/or the at least one enzyme capable of forming peptide bonds. Optionally, the mixing may include adding one or more co-factors, vitamins and/or minerals.

According to some embodiments, the process may include drying the hydrogel into a matrix powder capable of forming a hydrogel when hydrated. Optionally, the drying may include freeze drying, spray drying, and/or vacuum drying.

### BRIEF DESCRIPTION OF THE DRAWINGS

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIG. 1 illustratively depicts a process for production of the herein disclosed composition, in accordance with some embodiments.

FIG. 2 illustratively depicts a process for production of the herein disclosed composition in accordance with some embodiments.

FIG. 3 is an exemplary flow diagram of a process for production of a composition in accordance with some embodiments.

FIG. 4 is an exemplary Texture Profile Analysis (TPA) graph for a non-sticky material in accordance with some embodiments.

FIG. 5 is an exemplary Texture Profile Analysis (TPA) graph for a non-sticky and a sticky material in accordance with some embodiments.

FIG. 6 shows exemplary gel results analysis for hardness (N)—the highest peak force measured during first compression of the hereindisclosed hydrogel (MP) as compared to methyl cellulose (MC). in accordance with some embodiments.

FIG. 7 shows exemplary gel results analysis for cohesiveness of the hereindisclosed hydrogel (MP) as compared to methyl cellulose (MC). in accordance with some embodiments.

FIG. 8 shows exemplary gel results analysis for springiness of the hereindisclosed hydrogel (MP) as compared to methyl cellulose (MC). in accordance with some embodiments.

FIG. 9 shows exemplary gel results analysis for gumminess of the hereindisclosed hydrogel (MP) as compared to methyl cellulose (MC). in accordance with some embodiments.

FIG. 10 shows exemplary gel results analysis for chewiness of the hereindisclosed hydrogel (MP) as compared to methyl cellulose (MC). in accordance with some embodiments.

FIG. 11 is a graph comparing the gel results analysis for hardness (N), of the hereindisclosed hydrogel (MP) as compared to albumen, in accordance with some embodiments.

FIG. 12 is a graph comparing the gel results analysis for cohesiveness of the hereindisclosed hydrogel (MP) as compared to albumen, in accordance with some embodiments.

FIG. 13 is a graph comparing the gel results analysis for gumminess of the hereindisclosed hydrogel (MP) as compared to albumen, in accordance with some embodiments.

FIG. 14 is a graph comparing the gel results analysis for springiness of the hereindisclosed hydrogel (MP) as compared to albumen, in accordance with some embodiments.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout. In the figures, same reference numerals refer to same parts throughout.

According to some embodiments, there is provided a composition comprising a porous plant protein matrix comprising crosslinked plant derived polypeptides, and at least one enzyme capable of catalyzing amino acid oxidation and/or at least one enzyme capable of forming peptide bonds between amino acid residues, wherein the matrix may be capable of forming a hydrogel when hydrated. Preferably, the composition is devoid of synthetic gelling agents such as but not limited to methyl cellulose. According to some embodiments, the plant proteins is obtained from a plant protein source such as plant protein isolate, a plant protein concentrate or a plant protein flour.

Some embodiments of the present disclosure provide a combination of enzymes and physical treatment of plant-based proteins to give them meat, fish, dairy or egg protein like properties. According to some embodiments, the treatment may increase plant-based proteins crosslinking which in turn advantageously provides improved water retention, resulting in a juicier texture.

According to some embodiments, the composition may serve as a raw material for the plant-based protein substitute industry. According to some embodiments, the treated protein may advantageously be added to products without altering their existing production lines, essentially replacing methylcellulose and other added gelling agents. For example, the amount of the composition used may be between about 12-6% of the product, which is the same amount as the methylcellulose or other synthetic gelling agents in use today.

Hydrogels allow for water retention and crosslinking since a hydrogel is a three-dimensional (3D) network of hydrophilic polymers that swell in water and hold a large amount of water, while maintaining their structure, due to chemical or physical crosslinking of individual polymer chains. Hydrogels are currently mainly used in biomedical applications and are mostly made by synthetic processes. According to some embodiments, the herein disclosed plant-based hydrogel, based on a functional protein, may advantageously replace methylcellulose and other synthetic gelling agents. Optionally, the plant-based hydrogel may replace carboxymethylcellulose (CMC) and other synthetic gelling agents in alternative meat products. Optionally, the plant-based hydrogel may be used in other products, such as egg-alternative products, fish-alternative products, meat-alternative products, and dairy replacements. Optionally, the plant-based hydrogel may advantageously have egg-like properties, as opposed to methylcellulose and other synthetic gelling agents, which tend to be too jelly-like

According to some embodiments, multiple treated enzymes may be combined to create a gel that is stable during heating and/or binding of the material, without the need for methylcellulose, additives, flavor agents and/or stabilizers. According to some embodiments, the composition advantageously does not change its behavior when heated and/or cooled, i.e., it may be thermoresistant. According to some embodiments, the composition advantageously has a water retention capability at least as good as and even higher than those of cellulose derivatives. According to some embodiments, the composition may have advantageous hardness, springiness, chewiness and/or cohesiveness characteristics. According to some embodiments, the composition may have improved hardness, springiness, chewiness and/or cohesiveness, as compared to methylcellulose and/or its derivatives.

According to some embodiments, using enzymes advantageously shrinks the ingredient list since enzymes are considered a processing aid by regulations and therefore need not appear on the label. Enzymes may be used for protein modification, particularly for their incorporation into food systems, since the reactants and by-products are non-toxic. Moreover, enzymatic modification is environmentally friendly and less energy-consuming without production of toxic by-products. The modification may be achieved under mild condition with few by products. The reaction time is rapid due to the specificity of the enzymes.

Certain enzyme families are known for their crosslinking abilities and are considered a natural ingredient or processing aid. According to some embodiments, using multiple treated enzymes may allow for an overall improved flavor by eliminating compounds that may cause aftertaste. According to some embodiments, the enzymes may be Generally Recognized as Safe (GRAS) substances.

According to some embodiments, using multiple enzymatic groups simultaneously may lower the required concentration of each of them. Optionally, the effect of using multiple enzymatic groups simultaneously may provide a synergistic effect in terms of crosslinking capabilities and/or in terms of achieving the desired hardness, springiness, chewiness and/or cohesiveness. Optionally, using multiple enzymatic groups may reduce the overall production cost. Optionally, using different enzymatic groups enables achieving a desired structural stability and a similar texture to that found in animal products. Optionally, using an enzyme mixture, the texture that the raw material gives the final product may be modified and adapted by demand. According to some embodiments, using multiple enzymes in the production process is the ability to introduce new functionality, such as protein-pectin bonds. Optionally, addition of protein-pectin bonds may improve textural stability, increasing the product's water retention capacity and gelation properties.

The use of enzymes in highly viscous reactions has proven to be problematic. As viscosity increases, enzymatic activity decreases. Every enzyme used increases the mixture's viscosity, which theoretically should inhibit the use of additional enzymes. However, according to some embodiments, adjusting the reaction conditions enables use of multiple enzymatic groups to produce stable products with strong protein connections and a high water-retention capability, contributing to achieving good texture. Without being bound by theory, this may be achieved for example, by adding at least the cross-linking enzymes before the maximum viscosity is reached, the addition of the crosslinking enzymes may lead to an additional increase in the viscosity. Optionally, a balance may be found between enzymes such as hydrolases (e.g., pectinmethylesterase, cellulases, amylases, etc.) which decrease viscosity and the crosslinked enzymes which increase the viscosity. Optionally, the enzymes may be immobilized.

According to some embodiments, creating a combined matrix by using more than one enzymatic group may enable the production of texture and juiciness similar to those produced by animal proteins. Non-limiting examples of food products which may make use of the composition are listed in Table 1 below.

TABLE 1

| Products | Conc. (min-max) | Functional properties |
|---|---|---|
| Mayonnaise | 0.05-5% | Emulsion, stabilization |
| Ice cream | 0.05-10% | Emulsion, stabilization, taste, mouth feel |
| Bakery | 0.05-8% | Emulsion, stabilization, Improving swelling, water |
| | | retention, denaturation |
| Yeast Dough | 0.05-7% | Emulsion, stabilization, Improving swelling, water |
| | | retention, denaturation |
| Plant base meat/meat | 0.05-25% | Replacing methylcellulose, stabilizers, improving |
| alternative | | texture, adding plant base protein, denaturation, water |
| | | retention |
| Plant base | 0.05-25% | Replacing methylcellulose, stabilizers, improving |
| fish/fish | | texture, adding plant base protein, denaturation, water |
| alternative | | retention |
| Vegan Omelet | 0.05-25% | Replacing methylcellulose, stabilizers, improving |
| | | texture, adding plant base protein, denaturation, water |
| | | retention, taste |
| Vegan Fried egg | 0.05-25% | Replacing methylcellulose, stabilizers, improving |
| | | texture, adding plant base protein, denaturation, water |
| | | retention, taste |
| Energy bar | 0.05-20% | Replacing methylcellulose, stabilizers, improving |
| | | texture, adding plant base protein, denaturation, water |
| | | retention, taste |
| Dairy products | 0.05-10% | Replacing methylcellulose, stabilizers, improving texture, |
| | | adding plant base protein, denaturation, water retention |
| Chocolate | 0.05-15% | Replacing methylcellulose, stabilizers, improving texture, |
| paste/spreads | | adding plant base protein, denaturation, water retention |
| Cookies | 0.05-10% | Stabilizers, texture, mouth feel, body |
| Pasta | 0.05-10% | Stabilizers, texture, mouth feel, body |
| Animal feed | 0.05-15% | Replacing methylcellulose, stabilizers, improving texture, |
| | | adding plant base protein, denaturation, water retention |
| Nutrition food | 0.05-7% | Replacing methylcellulose, stabilizers, improving texture, |
| | | adding plant base protein, denaturation, water retention |
| Sport nutrition | 0.05-25% | High protein, replacing methylcellulose, stabilizers, |
| products | | improving texture, adding plant base protein, |
| | | denaturation, water retention |
| Salty snacks | 0.05-25% | High protein, replacing methylcellulose, stabilizers, |
| | | improving texture, adding plant base protein, |
| | | denaturation, water retention |
| Aioli spreads and | 0.05-10% | Emulsion, stabilization, Improving swelling, water |
| sauces | | retention, denaturation |
| Cosmetic | 0.05-20% | Emulsion, stabilization, water retention, denaturation |
| composition | | |
| Nutraceuticals | 0.05-20% | Emulsion, stabilization, water retention, denaturation |

According to some embodiments, exposing the buried amino acids in a protein and/or polypeptide with a unique composition of enzymes and processes may produce ready to use protein with a porous structure. According to some embodiments, the presence and/or accessibility of the target amino acid side chains may depend on the conformation of the substrate polypeptide and/or protein, which may be an important factor affecting formation of intermolecular and/or intramolecular crosslinks in polypeptides and/or proteins. According to some embodiments, the polypeptide and/or protein may undergo preprocessing. Optionally, the preprocessing may include thermal treatment of a polypeptide and/or protein solution. Optionally, and without being bound by any theory, as a consequence of the preprocessing, the polypeptide chains unfold and, internal sulfhydryl groups, hydrophobic side chains and/or any other previously buried active sites in the core of the native-state structure, may become more exposed for enzymatic reaction. Optionally, enzymatic crosslinking may provide bonds, optionally covalent bonds, between protein and/or polypeptide chains under mild conditions and/or result in reactive compounds that may optionally polymerizes and/or lead to covalent crosslinking spontaneously.

According to some embodiments, the composition may comprise semi-activated plant derived polypeptides. Optionally, the semi-activated plant derived polypeptides may be ready to use on the production line, thereby providing shorter production time. Optionally, the semi-activated plant derived polypeptides may be combined with additional compounds for use in a food product (e.g., additional protein, co-factors, vitamins, minerals, enzymes, sugars, fats, fiber (e.g., fibers from rose hip, pear, apple, guava, quince, plum, gooseberry, citrus fruit, etc., etc.), etc. or any combination thereof). Optionally, the semi-activated plant derived polypeptides may be a homogeneous mass. Optionally, the semi-activated plant derived polypeptides may have widespread industry usage (e.g., not limited to only one or two types of protein).

According to some embodiments, the composition may have a better effect on gut microbiome.

According to some embodiments, the composition may include one or more plant proteins and one or more enzymes. Optionally, the one or more enzymes may crosslink the plant protein. Optionally, the crosslinked plant protein may result in a porous plant protein matrix. Optionally, the porous plant protein matrix may form a hydrogel when hydrated.

As used herein, the term “porous” refers to a material having many small holes (pores) that allow air or liquid to pass through them more readily than non-porous materials, which have a much tighter cell structure preventing ease of flow. For example, glass, metal, plastic, and varnished wood are examples of non-porous materials, while untreated wood, drapes, carpet, membranes, and cardboard are porous.

As used herein, the term “protein matrix” refers to large assemblies of tightly bound proteins forming an extensive network.

As used herein, the term “plant derived” refers to made from a plant, wherein the plant may be a fungus, cactus, herbaceous plant, flowering plant, food crop plant, and/or combinations thereof. For example, the plant derived material may be made from or extracted from any part of a plant, such as a root, stem, leaf, seed, flower, fruit plant, and/or combinations thereof. According to some embodiments, the term “derived” may be substituted with the term “isolated”.

As used herein, the term “polypeptide” refers to a continuous, unbranched chain of amino acids joined by peptide bonds. A peptide consisting of 2 or more amino acids. Peptides differ from polypeptides in that they are made up of shorter chains of amino acids (at least 10 amino acids). Amino acids make up polypeptides which, in turn, make up proteins. For example, amalin, glucagon, etc.

As used herein, the term “protein” refers to long chains of amino acids held together by peptide bonds, a protein may contain one or more polypeptides. For example, amylase, lipase, pepsin, hemoglobin, insulin, tubulin, keratin, etc.

As used herein, the term “enzyme” refers to a biological catalyst which speeds up the rate of a specific chemical reaction in an organism, and is almost always a protein. For example, transglutaminase (EC 2.3.2.13), pectinmethylesterase (EC 3.1.1.11), laccase (EC 1.10.3.2), amylase (EC 3.2.1.X), cellulases (EC 3.2.1.4), lipase (EC 3.1.1.X), tyrosinase (EC 1.14.18.1), oxidoreductase, peroxidase (EC 1.11.1.X), sulfhydryl oxidase glutathione oxidase (EC 1.8.3.3), sortase A (EC3.4.22.70), pectin lyase (EC 4.2.2.10), polygalacturonase (EC 3.2.1.15), transferase (EC 2.1 to EC 2.10), hydrolase (EC 3.1 to EC 3.13), etc.

As used herein, the term “hydrogel” refers to a water-insoluble, three-dimensional (3D) network of hydrophilic polymers that can swell in water and hold a large amount of water, while maintaining the structure due to chemical or physical crosslinking of individual polymer chains. For example, gelatin, collogen, alginate etc.

As used herein, the term “hydrated” refers to chemically combining with water in its molecular form. Hydration involves the addition of water from a molecule, ion or substance. Dehydration involves the removal or loss of water from a molecule, ion or substance. Rehydration involves the return of water to a dehydrated molecule, ion or substance.

According to some embodiments, the composition is devoid of methylcellulose and other synthetic gelling agents.

According to some embodiments, a protein may be activated by exposing the buried active functional amino acids residues. Optionally, the amino acids may undergo enzymatic crosslinking using a combination of enzymes and process. Optionally, this process may result in a semi-activated protein with a porous structure.

According to some embodiments, the plant protein may be derived from at least one of pea, corn, wheat, rice, nuts, almond, peanut, seitan, lentil, chickpea, flaxseed, chia seed, oat, buckwheat, bulgur, millet, sunflower, canola, legumes, pulses, tofu, soy, tempeh, seitan, seeds, grain, chickpeas, lentils, legume, lupin, rapeseed, yeast, algae, microalgae, edamame, spelt, teff, hemp seeds, spirulina, amaranth, quinoa, leafy vegetables, oats, wild rice, chia seeds, fava bean, yellow pea, mung bean, nuts, protein-rich fruits and vegetables (such as broccoli, spinach, asparagus, artichokes, potatoes, sweet potatoes, brussels sprouts, sweet corn, guava, cherimoyas, mulberries, blackberries, nectarines, bananas, etc.) or any combination thereof. Each possibility is a separate embodiment.

According to some embodiments, the plant protein may selected from at least one of leghemoglobin, non-symbiotic hemoglobin, hemoglobin, myoglobin, chlorocruorin, erythrocruorin, neuroglobin, cytoglobin, protoglobin, truncated 2/2 globin, HbN, cyanoglobin, HbO, Glb3, and cytochromes, Hell's gate globin I, bacterial hemoglobins, ciliate myoglobins, flavohemoglobins, ribosomal proteins, actin, hexokinase, lactate dehydrogenase, fructose bisphosphate aldolase, phosphofructokinases, triose phosphate isomerases, phosphoglycerate kinases, phosphoglycerate mutases, enolases, pyruvate kinases, glyceraldehyde-3-phosphate dehydrogenases, pyruvate, decarboxylases, actins, translation elongation factors, ribulose-1,5-bisphosphate carboxylase oxygenase (rubisco), ribulose-1,5-bisphosphate carboxylase oxygenase activase (rubisco activase), albumins, glycinins, conglycinins, globulins, vicilins, conalbumin, gliadin, glutelin, gluten, glutenin, hordein, prolamin, phascolin (protein), proteinoplast, secalin, extensins, triticeae gluten, zein, any seed storage protein, oleosins, caloleosins, steroleosins or other oil body proteins, vegetative storage protein A, vegetative storage protein B, moong seed storage 8S globulin, etc., or derivatives thereof. Each possibility is a separate embodiment. According to some embodiments, the one or more plant proteins may be completely crosslinked or semi-crosslinked. According to some embodiments, the functional properties of plant-derived polypeptides may be altered by modifying the natural crosslinks or introducing new crosslinks into the structure of the polypeptide. Optionally, crosslinking may be due to peptide bonds between amino acid residues. Optionally, crosslinking may be due to amino acid oxidation.

According to some embodiments, crosslinking may be performed by at least one enzyme capable of catalyzing amino acid oxidation. Optionally, the enzyme capable of catalyzing amino acid oxidation may be an oxidoreductase (EC 1). Optionally, the oxidoreductase may be a multi-copper enzyme capable of oxidating phenolic residues. Optionally, a multi-copper enzyme may catalyze oxidation of a wide variety of phenolic compounds by a single electron removal mechanism, which results in the formation of free radicals with concomitant reduction of molecular oxygen to water. Optionally, the oxidoreductase may use H₂O₂ as an electron acceptor to oxidize a variety of organic and inorganic substrates, such as phenols, as a result of oxidation, a radical is formed that can react further with other substrates. Optionally, the oxidoreductase may be a laccase, a tyrosinase, a peroxidase, a glutathione oxidase or any combination thereof.

According to some embodiments, crosslinking may be performed by at least one enzyme capable of forming peptide bonds between amino acid residues. For example, the enzyme may accelerate the formation of isopeptide bonds between the side chains of glutamine residues and the side chains of lysine residues, thus enabling the formation of stable structures. Optionally the at least one enzyme capable of forming peptide bonds between amino acid residues may be a transferase (EC 2) or a peptidase (EC 3). Optionally, the transferase may be an amino-acyltransferases, such as a protein-glutamine gamma-glutamyltransferase. Optionally, the peptidase may be a cysteine endopeptidase.

According to some embodiments, the at least one enzyme capable of catalyzing amino acid oxidation and/or the at least one enzyme capable of forming peptide bonds between amino acid residues may be reversibly inactivated by drying and/or freezing. Optionally, the reversibly inactive enzyme may be reactivated upon hydration and/or thawing.

According to some embodiments, the composition may include an enzyme capable of hydrolyzing polysaccharides. Optionally, the enzyme capable of hydrolyzing a polysaccharide may be a pectinase (EC 3.2), an amylase (EC 3.2.1.1), a cellulase (EC 3.2.1.4) or any combination thereof. Optionally, pectinase may be selected from the group including pectolyase, pectozyme, and/or polygalacturona.

According to some embodiments, polysaccharides such as pectin (e.g., fibers from rose hip, pear, apple, guava, quince, plum, gooseberry, citrus fruit, etc.) may be available for the formation of safe and nontoxic hydrogel materials. Optionally, the pectin in the final product may undergoes extrusion and addition to plant-based protein. Optionally, protein-pectin binding may produce a stable form that improves water retention and/or gel formation. Optionally, protein-pectin binding may improve the texture and/or taste of a plant-based product. Optionally, enzymatic crosslinking may allow for stable protein-protein binding and protein-pectin binding without external stabilizers.

According to some embodiments, the composition may include a lipase. Optionally, the lipase may be selected from a phospholipase (E.C. 3.1.1.4), a lysophospholipase (EC: 3.1.1.5), a galactolipase (EC 3.1.1.26), a feruloyl esterase (EC 3.1.1.73) or any combination thereof.

According to some embodiments, lipase may have high selectivity toward transesterification/esterification/hydrolysis of saturated fatty acids, mono, di-and tri-unsaturated fatty acids, as free fatty acids and/or in the form of fatty acyl groups, and low selectivity toward the transesterification/esterification/hydrolysis of n−3 fatty acids as free fatty acids or as fatty acyl groups Optionally, addition of a lipase may produce free fatty acids which may affect the flavor, aroma and/or the shelf life of the various food products produced.

According to some embodiments, the plant protein matrix may include a mediator. Optionally, the mediator may mediate crosslinking of the polypeptides. For example, Scheme 1 is an exemplary reaction scheme showing crosslinking of proteins in the absence of a mediator, while Scheme 2 is an exemplary reaction scheme showing crosslinking of proteins in the presence of a mediator.

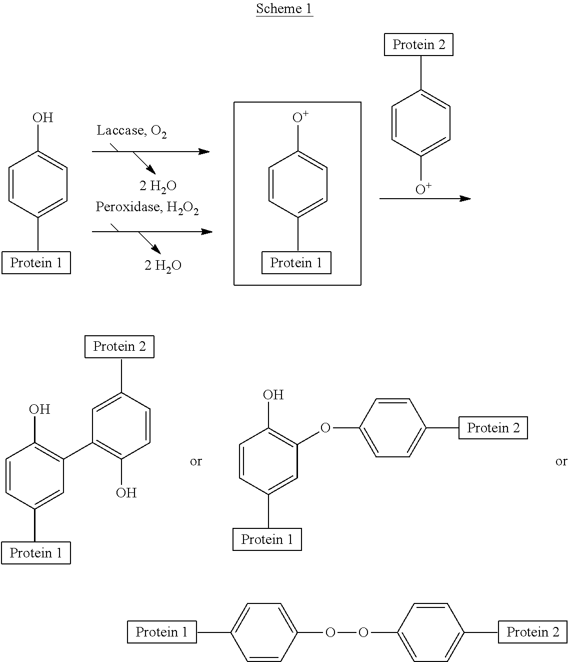

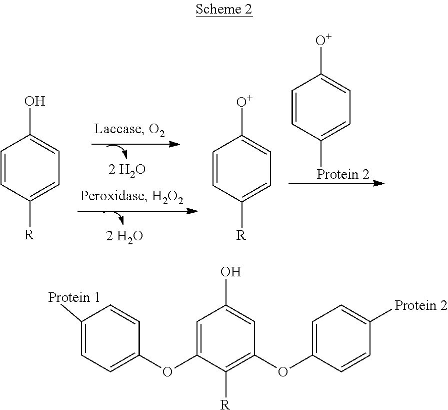

According to some embodiments, a mediator may be a small molecule which may be readily oxidized by enzymes, such as laccase, to produce radicals which may then react with a target substrate. Optionally, the unique enzyme composition may enable generation of mediators which may penetrate the exposed active site and assist the crosslinking enzymes. Optionally, a mediator may be a phenolic compound, such as monophenols, diphenols, etc. Optionally, sugar beet pectin (SBP) may be a source of a phenolic mediator (e.g., ferulic acid). Optionally, vanillin, vanillic acid, caffeic acid, catechin may be used as mediators. Each possibility is a separate embodiment.

According to some embodiments, the composition may include one or more co-factors, vitamins, minerals or combination thereof.

According to some embodiments, a cofactor may be a non-protein chemical compound or metallic ion that is required for an enzyme's role as a catalyst. Optionally, cofactors may be divided into two types: inorganic ions and complex organic molecules called coenzymes. Optionally, coenzymes may be derived from vitamins and/or other organic essential nutrients in small amounts. Optionally, a co-factor may be selected from flavin, heme, thiamine, folic acid, metal ions such as iron, magnesium, manganese, cobalt, copper, zinc, and molybdenum, iron-sulfur clusters, etc. and/or combinations thereof.

According to some embodiments, a vitamin may be an organic compound that is essential for biological activity. Optionally, a vitamin may be selected from the group including vitamins A, C, D, E, and K, choline, and the B vitamins (thiamin, riboflavin, niacin, pantothenic acid, biotin, vitamin B6, vitamin B12, and folate/folic acid), etc. and/or combinations thereof.

According to some embodiments, a mineral may be a macromineral and/or a trace mineral. Optionally, a macromineral may be selected from the group including calcium, phosphorus, magnesium, sodium, potassium, chloride, sulfur, etc. and/or combinations thereof. Optionally, a trace mineral may be selected from the group including iron, manganese, copper, iodine, zinc, cobalt, fluoride, selenium etc. and/or combinations thereof.

According to some embodiments, a mixture of enzymes may provide a synergistic effect. Optionally, the synergistic effect may allow for a reduction in the total enzyme concentrations required. Optionally, multiple enzymes may be added sequentially and/or simultaneously.

According to some embodiments, using a mixture of enzymes may lower the concertation required of each of them due to the synergistic effect, thereby reducing the overall production cost. Optionally, each enzyme may be used in a concentration of up to about 0.1% w/w, about 0.25% w/w, about 0.5% w/w or about 1% w/w. Each possibility is a separate embodiment.

According to some embodiments, the composition may be in the form of a powder, a solid, a hydrogel and/or a mixture thereof. Optionally, the composition may be freeze-dried. Optionally, the porous plant protein matrix may form a hydrogel when hydrated. Optionally, the hydrogel may be thermoresistant. Optionally, thermo-resistance may be a change of less than about 25%, less than about 20%, less than about 15%, less than about 10%, less than about 5%, or less than about 1% upon heating/cooling.

According to some embodiments, the springiness of the hydrogel may change by less than 20%, less than 15%, less than 10% or less than 5% when cooled/heated. Each possibility is a separate embodiment. According to some embodiments, the hardness of the hydrogel may change by less than 20%, less than 15%, less than 10% or less than 5% when cooled/heated. Each possibility is a separate embodiment. According to some embodiments, the gumminess of the hydrogel may change by less than 20%, less than 15%, less than 10% or less than 5% when cooled/heated. Each possibility is a separate embodiment. According to some embodiments, the cohesiveness of the hydrogel may change by less than 20%, less than 15%, less than 10% or less than 5% when cooled/heated. Each possibility is a separate embodiment. Optionally, the heating may be to about 50-80° C. for at least 2 min and the cooling may be room temperature and/or 4° C.

According to some embodiments, the hydrogel may comprise active residues which upon rehydration may enable crosslinking between the plant-based peptide matrix and one or more externally added polypeptides and/or proteins. According to some embodiments, the concentration of the plant derived protein in the hydrogel may be less than about 30% w/w, less than about 20% w/w or less than about 10% w/w. Each possibility is a separate embodiment. According to some embodiments, the concentration of the plant derived protein in the hydrogel may be about 5-30% w/w or 10-30% w/w. According to some embodiments, the hydrogel comprises at least about 60% w/w, at least about 70% w/w, at least about 80% w/w or at least about 90% w/w water.

According to some embodiments, the composition may comprise about 0.02 to about 0.08% w/w salt. Optionally, the salt may be added by admixing. Optionally, the salt may be selected from the group including sodium chloride, or any other sodium salts, potassium salts, calcium salts, magnesium salts, sodium citrate and a combination thereof. Each possibility is a separate embodiment.

According to some embodiments, the salt may serve as a co-factor the enzymes.

According to some embodiments, the composition may be dehydrated to form a solid. Optionally, the solid may be milled to form a powder. Optionally, the powder may have a particle size distribution of between about 5 μμm to about 5 mm, between about 50 μm to about 1 mm, or between about 0.1 to about 0.5 mm. Each possibility is a separate embodiment.

According to some embodiments, a food product may include the composition. Optionally, the composition may comprise semi-activated plant derived polypeptides. Optionally, the composition may form crosslinks between the semi-activated plant derived peptide matrix and one or more externally added polypeptides and/or proteins. Optionally, the food product may include between about 1-50% w/w, between about 1-25% w/w, or between about 1-15% w/w of the composition. Each possibility is a separate embodiment.

According to some embodiments, the food product may be a plant-based meat alternative product, plant-based fish alternative product, egg-less egg alternative product, a dairy replacement product, a chocolate alternative product, an egg-less bakery product, a hybrid meat-plant-based meat alternative product, a hybrid fish-plant-based fish alternative product, a hybrid dairy-plant-based dairy alternative product, a hybrid egg-plant-based egg alternative product, or a combination thereof. Each possibility is a separate embodiment.

According to some embodiments, the change in the cohesiveness before and after cooking of the food product may be about 20% less, about 15%, less, about 10% or less or about 5% less than for food products including the hereindisclosed hydrogel as compared to the change in the cohesiveness of the same food product including methyl cellulose as a gelling agent. Each possibility is a separate embodiment.

According to some embodiments, the change in the hardness before and after cooking of the food product may be about 20% lesser, about 15%, less, about 10% less or about 5% less than for food products including the hereindisclosed hydrogel as compared to the change in the cohesiveness of the same food product including methyl cellulose as a gelling agent. Each possibility is a separate embodiment.

According to some embodiments, the change in the springiness before and after cooking of the food product may be about 20% less, about 15% less, about 10% less or about 5% less than for food products including the hereindisclosed hydrogel as compared to the change in the cohesiveness of the same food product including methyl cellulose as a gelling agent. Each possibility is a separate embodiment.

According to some embodiments, the change in the chewiness before and after cooking of the food product may be about 20% less, about 15% less, about 10% less or about 5% less than for food products including the hereindisclosed hydrogel as compared to the change in the cohesiveness of the same food product including methyl cellulose as a gelling agent. Each possibility is a separate embodiment.

According to some embodiments, a process for producing a porous plant protein matrix capable of forming a hydrogel when hydrated may include mixing plant-derived polypeptides with at least one enzyme capable of catalyzing amino acid oxidation and/or at least one enzyme capable of forming peptide bonds between amino residues, and incubating at conditions allowing crosslinking of at least a portion of the plant derived polypeptides, thereby forming a hydrogel. Optionally, the incubation conditions for crosslinking may include maintaining the temperature below about 90° C., below about 80° C., below about 70° C., below about 60° C., below about 50° C., below about 45° C., below about 40° C., below about 35° C., below about 30° C., below about 25° C., below about 20° C., below about 15° C., below about 10° C., below about 5° C. for an extended period of time. Optionally, the extended period of time may be at least about 30 mins, at least about 1 hr, at least about 2 hrs, at least about 3 hrs, at least about 4 hrs, at least about 5 hrs, at least about 6 hrs, at least about 7 hrs, at least about 8 hrs, at least about 9 hrs, at least about 10 hrs, at least about 11 hrs, at least about 12 hrs, at least about 13 hrs, at least about 14 hrs, at least about 15 hrs, at least about 20 hrs, or at least about 24 hrs.

According to some embodiments, the process may include a step of preprocessing the plant-based polypeptides prior to and/or during the mixing to expose amino acid residues. Optionally, the preprocessing may include heating, pressure, sonication or any combination thereof Optionally, the preprocessing may include heating, pressure, sonication, extrusion, cold plasma, ultrasound, ultraviolet or any combination thereof. Optionally, ultrasound treatment may include sonication. Optionally, heating may include conventional heating, ohmic heating, microwave heating, radiofrequency heating, and/or infrared heating. Optionally, heating may be at a high temperature for short time, and/or mild temperature for long period, e.g., about 80-90°C. for 3-30 min, about 40 to about 60° C. for about 3 or less to about 9 hours, about 80-95° C. for 1 hour, etc. Optionally, the heating may be carried without or without mixing. Optionally, high pressure treatment may be static and/or dynamic. Optionally, extrusion may include thermo-mechanical processes, which combine high heat, high shear, and high pressure to cause cooking, sterilization, drying, melting, conveying, kneading, puffing texturizing, and/or forming of a food product. Optionally, cold plasma treatment may create a state of matter that contains a cocktail of reactive oxygen species, reactive nitrogen species (O·, ·OH, N·, HO²·, N₂*, N*, OH⁻, O²⁻, O⁻, O²⁺, N2⁺, N⁺, NO, O⁺, O₃, and/or H₂O₂) and ultraviolet radiations generated when the energy supplied to a gaseous environment dissociates the gas molecular bonds into fully or partially ionized gases (plasma). Optionally, the energy discharge source may be electrical, thermal, optical, electromagnetic, etc. Each possibility is a separate embodiment.

According to some embodiments, the process may include adding at least one enzyme capable of degrading polysaccharides to the plant-derived polypeptides prior to the mixing with one or more enzymes capable of catalyzing amino acid oxidation and/or the at least one enzyme capable of forming peptide bonds. Optionally, the amount of the at least one enzyme capable of degrading polysaccharides may be in the range of about 0.01-1% w/w.

According to some embodiments, the process may include a step of generating a semi-activated enzyme mixture. Optionally, the mixture may include one or more enzyme capable of catalyzing amino acid oxidation and/or one or more enzyme capable of forming peptide bonds and one or more enzymes capable of degrading polysaccharides.

According to some embodiments, the mixing may include adding one or more co-factors, vitamins and/or minerals.

According to some embodiments, the process may include drying the composition to a matrix powder capable of forming a hydrogel when hydrated. Optionally, the drying may include freeze drying, spray drying, vacuum drying, centrifuging, pressing, lyophilizing, hot air drying, drying under hot inert gases, screen mash, and/or any methods suitable to remove water or fluids and combination thereof

According to some embodiments, the hereto disclosed composition may be combined with other edible ingredients to form a food product, e.g., an artificial meat product which mimics one or more physical characteristics and/or functional properties of meat, such as texture, flavor, aroma, and/or appearance. Optionally, such other ingredients may be selected from apple cider, apple cider vinegar, baking powder, baking soda, beans, beef, beet juice, beet powder, black pepper, brown sugar, butter, canola oil, caramel, carrot fiber, carrots, cashews, cheese, chicken, chocolate, citrus, citrus extract, coconut oil, condensed milk, dairy, egg, egg substitute, fish, flour, garbanzo bean, garlic powder, honey, liquid smoke, maple syrup, margarine, monosodium glutamate, mustard powder, oil, olive oil, onion powder, paprika, pork, potato, potato starch, rice flour, salt, sodium benzoate, soy (protein and/or oil), soy sauce, spices, spirulina, sugar, sunflower oil, tomato juice, tomato powder, tomato sauce, tomatoes, turmeric, vanilla, vinegar, vitamins and minerals, walnuts, water, wheat, wheat flour, wheat gluten, xanthan gum, yeast, yeast extract, etc. and/or combinations thereof.

A TPA test is a 2-cycle (two bite) compression test with a time delay between the cycles. The sample is usually bite sized (e.g., 1 cm³) and the deformation is typically between about 75% to about 90% of the height to simulate chewing by teeth. The test was originally developed by Friedman and Szczesniak at the General Foods Corporation, and was later modified by Malcolm Bourne wherein some parameters were slightly amended.

A TPA test may be used to calculate or determine to test a variety of parameters characteristic of the sample, e.g., hardness, cohesiveness, springiness, gumminess, chewiness, resilience, stickiness, adhesiveness, stringiness, etc.

Resilience is a measurement of how the sample recovers from deformation and is not a parameter from the original Texture Profile Analysis concept.

It is the ratio of the work (area under the curve) given back by the sample during the first release divided by the work absorbed by the sample during the first compression, i.e.

ti Area 4/Area 3

Stickiness is the minimum peak force during the first compression cycle (first bite)—Refers to the Soft “Sticky” Material” graph, i.e., Peak force in negative region

Adhesiveness is the negative work (area under the curve) for the first bite so is the work required to overcome the attractive forces between the food and the compression plates—Refer to the Soft “Sticky” Material” graph, i.e.

Work done in negative region=A3 in the second graph type

Stringiness is the distance the product is extended during decompression before separating from the compression probe and is not a parameter from the original Texture Profile Analysis concept.

Additionally, the parameters may be physical and/or sensory (e.g., while chewing), for examples see Table 2 below.

TABLE 2

| | Physical | Sensory |
|---|---|---|
| Hardness | Force necessary to attain a given deformation | Force required to compress a substance |
| | | between molar teeth (in the case of |
| | | solids) or between tongue and palate |
| | | (in the case of semi-solids). |
| Cohesiveness | Extent to which a material can | Degree to which a substance is |
| | be deformed before it ruptures. | compressed between the teeth before it |
| | | breaks. |
| Springiness | Rate at which a deformed | Degree to which a product returns to its |
| | material goes back to its | original shape once it has been |
| | undeformed condition after the | compressed between the teeth |
| | deforming force is removed | |
| Chewiness | Energy required to masticate a | Length of time (in sec) required to |
| | solid food to a state ready for | masticate the sample, at a constant rate |
| | swallowing: a product of | of force application, to reduce it to a |
| | hardness, cohesiveness and | consistency suitable for swallowing |
| | springiness | |

According to some embodiments, the food product cohesiveness of the food product changes by less than about 5%, less than about 10%, or less than about 15% before and after cooking.

According to some embodiments, the food product hardness of the food product changes by less than about 5%, less than about 10%, or less than about 15% before and after cooking.

According to some embodiments, the food product springiness of the food product changes by less than about 5%, less than about 10%, or less than about 15% before and after cooking.

According to some embodiments, the food product chewiness of the food product changes by less than about 5%, less than about 10%, or less than about 15% before and after cooking.

Reference is now made to the figures.

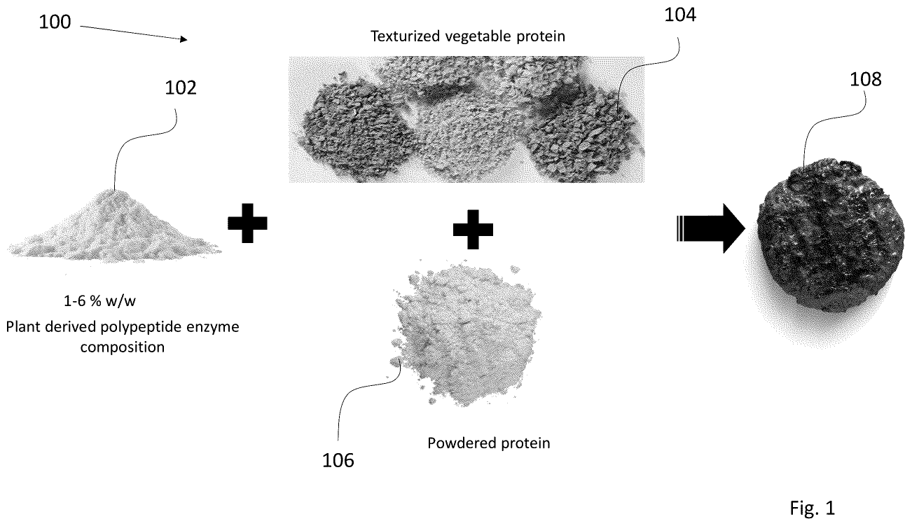

FIG. 1 shows an exemplary process for producing a composition in accordance with some embodiments. For example, in the process 100, 1-6% w/w of a protein-enzyme matrix 102 may be mixed with a plant derived polypeptide to form the hereindisclosed matrix (in the form of a powder or as hydrogel). The matrix 102 may then be mixed with powdered protein 106 and/or texturized vegetable protein (TVP) 104. Optionally, additional enzymes, co-factors, vitamins, minerals, and/or combinations thereof may be added to produce a plant derived polypeptide enzyme composition for use as or in a food product 108.

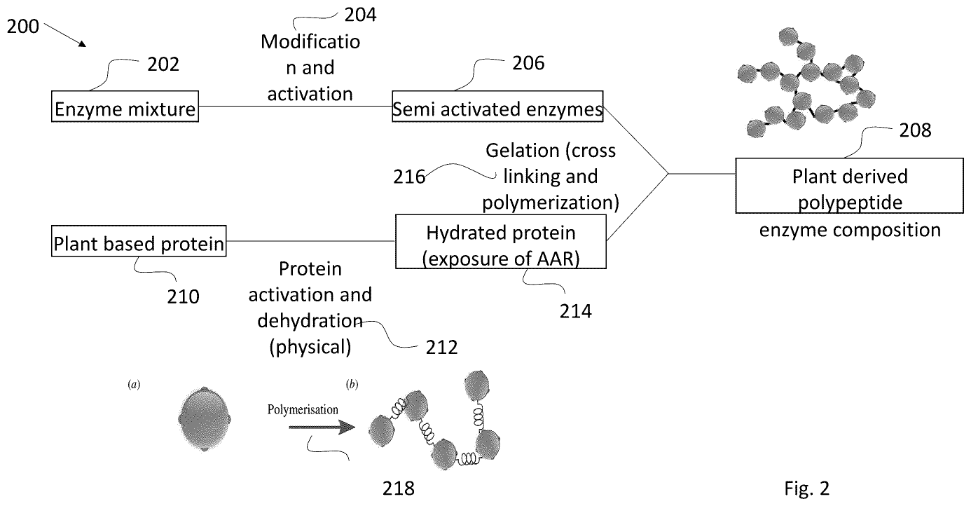

FIG. 2 is a schematic diagram of a process for production of composition in accordance with some embodiments. For example, in the process 200, an enzyme mixture 202 may undergo combination, modification, treatment and/or activation 204 to produce semi-activated enzymes 206. Plant-based proteins 210 may be activated and/or dehydrated 212 e.g., to produce powdered protein and/or texturized vegetable protein, which may then be hydrated to expose the amino acid residues (AAR) 214. The enzymes 206 may then be added to the protein 212. Gelation (crosslinking and polymerization) 216 of the hydrated plant-based protein 214 with the semi-activated enzymes 206 produces the protein-enzyme matrix, which may be hydrated to form a hydrogel 208. Polymerization of amino acids (a) or peptides to produce polypeptides (b) 218 may produce synthetic plant-based polypeptides.

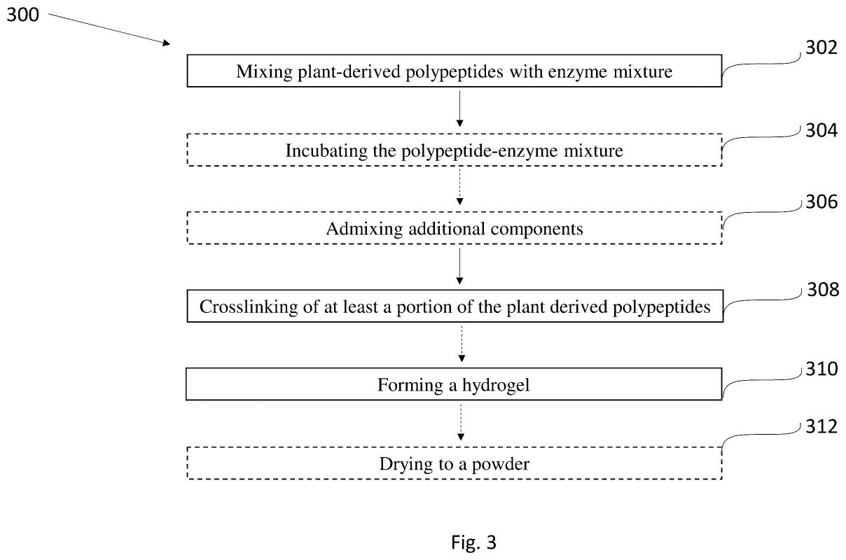

FIG. 3 is an exemplary flow diagram of a process for production of a composition in accordance with some embodiments. For example, in the process 300 mixing 302 plant-based polypeptides with water and an enzyme mixture comprising at least one enzyme capable of catalyzing amino acid oxidation and/or at least one enzyme capable of forming peptide bonds between amino acid residues. The polypeptide-water mixture is optionally heated to cause exposure of buried residues in the polypeptide, followed by cooling to a temperature optimal for the enzymatic reaction. According to some embodiments, the protein to enzyme ratio is in a range of 1:0.005-1:0.1. Optionally, in step 304 the polypeptide-enzyme mixture may be incubated at a non-reactive temperature prior to proceeding., As a further option, in step 306 additional components such as cofactors, salts, nutrients, minerals, fibers, etc., may be admixed. In step 308 at least a portion of the plant derived polypeptides is crosslinked to form the protein-enzyme matrix by incubating the polypeptide and the enzyme mixture at a temperature suitable for the reaction, thereby forming a hydrogel (Step 310). The hydrogel may optionally be dried 312 to form a powder, which can be reconstituted into a hydrogel when hydrated.

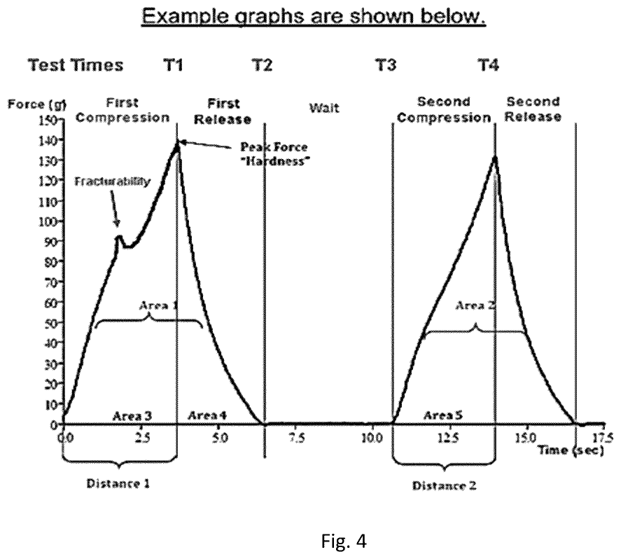

FIG. 4 is an exemplary Texture Profile Analysis (TPA) graph for a non-sticky material in accordance with some embodiments. From the various regions on the graph parameters characteristic of materials such as hardness, cohesiveness, springiness, gumminess, chewiness, resilience, stickiness, adhesiveness, stringiness, etc. may be derived.

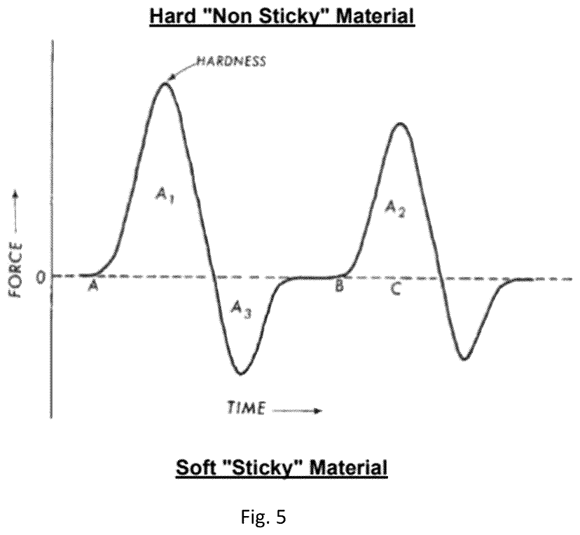

FIG. 5 is an exemplary Texture Profile Analysis (TPA) graph for a non-sticky and sticky material in accordance with some embodiments. For example, non-sticky materials give peaks above the x-axis and sticky materials give peaks below the x-axis.

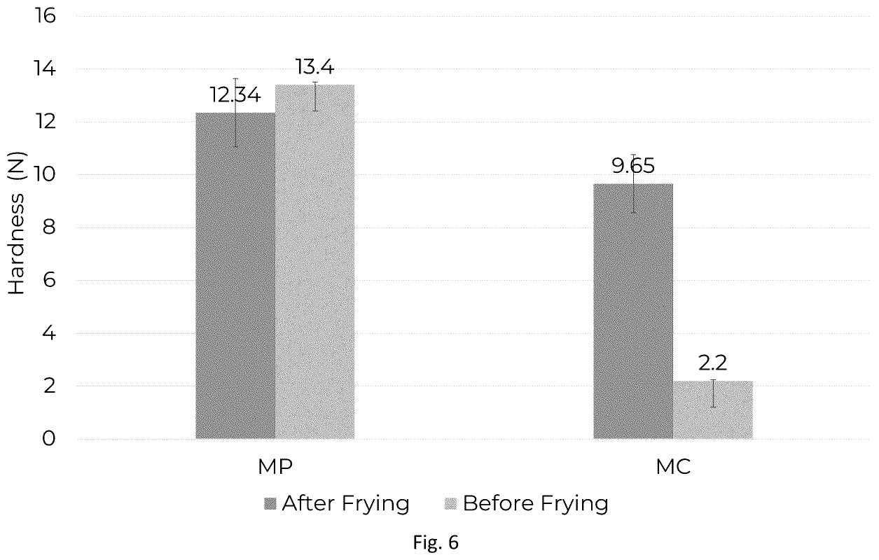

FIG. 6 is a graph comparing the gel results analysis for hardness (N), defined as the highest peak force measured during first compression, in accordance with some embodiments. Hardness is the physical force necessary to attain a given deformation.

In sensory terms, this is the force required to compress a substance between molar teeth (in the case of solids) or between tongue and palate (in the case of semi-solids). In a TPA test, this is the maximum peak force during the first compression cycle (first bite) and has often referred to as firmness. Additionally, fracturability (originally called brittleness) is the force at the first significant break in the TPA curve (if present).

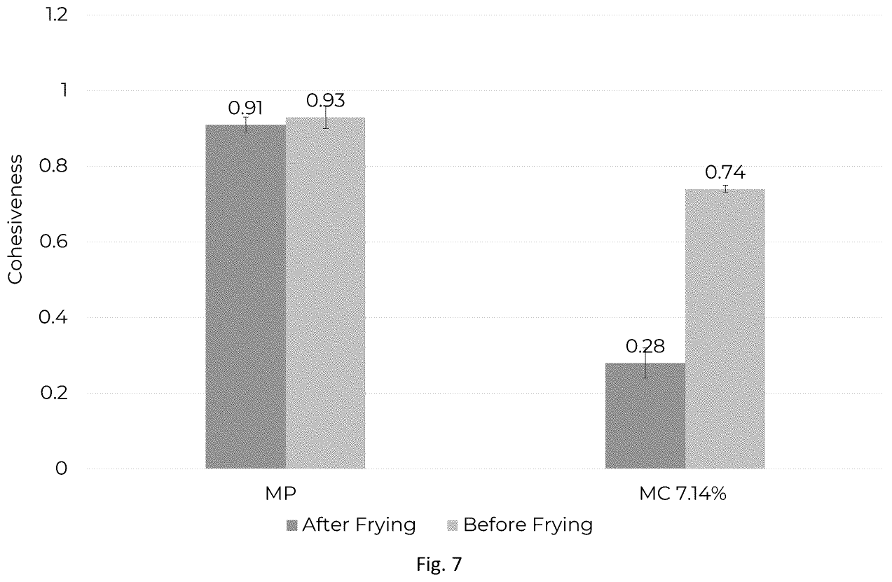

FIG. 7 is a graph comparing the gel results analysis for cohesiveness in accordance with some embodiments. Cohesiveness is defined as the extent to which a material can be deformed before it ruptures. In sensory terms, it is the degree to which a substance is compressed between the teeth before it breaks. In a TPA curve, this is the ratio of the work (area under the curve) during second compression divided by the work during first compression, i.e.

Area 2/Area 1

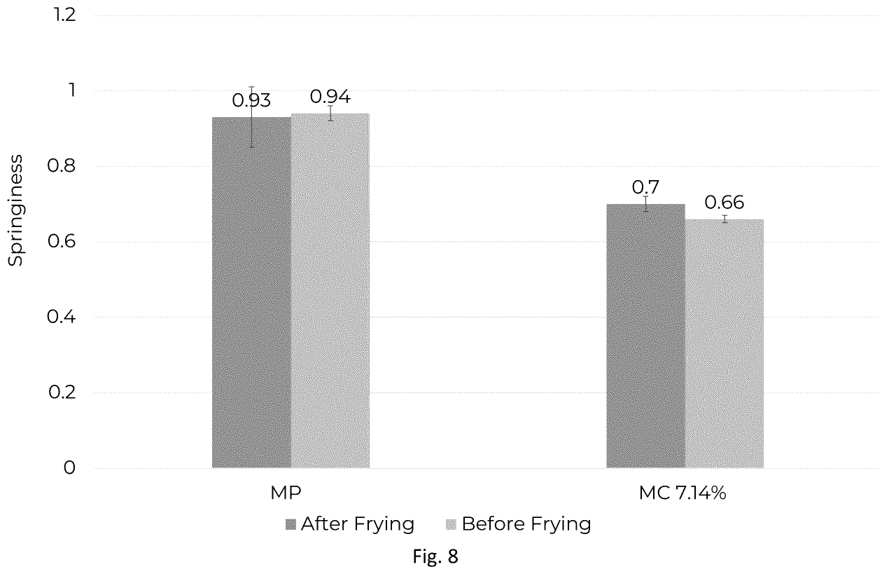

FIG. 8 is a graph comparing the gel results analysis for springiness in accordance with some embodiments. Springiness is the rate at which a deformed material returns to its undeformed condition after the deforming force is removed. In sensory terms, this is the degree to which a product returns to its original shape once it has been compressed between the teeth. In a TPA curve, this is the permanent compression of the sample after the first cycle, i.e., difference

Distance 2/Distance 1

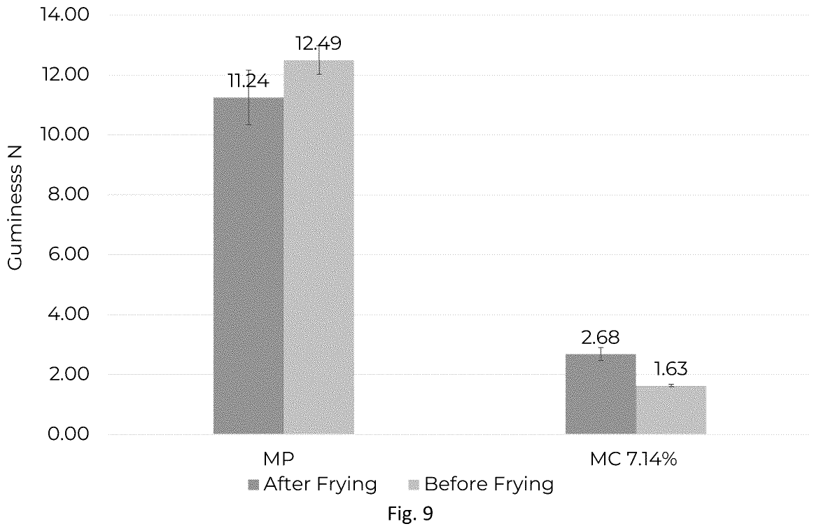

FIG. 9 is a graph comparing the gel results analysis for gumminess in accordance

with some embodiments. In a TPA curve, gumminess is reported for semisolids and is the product of Hardness*Cohesiveness, i.e.

$$Hardness * ( Area 2 / Area 1 ) = Hardness * Cohesiveness$$

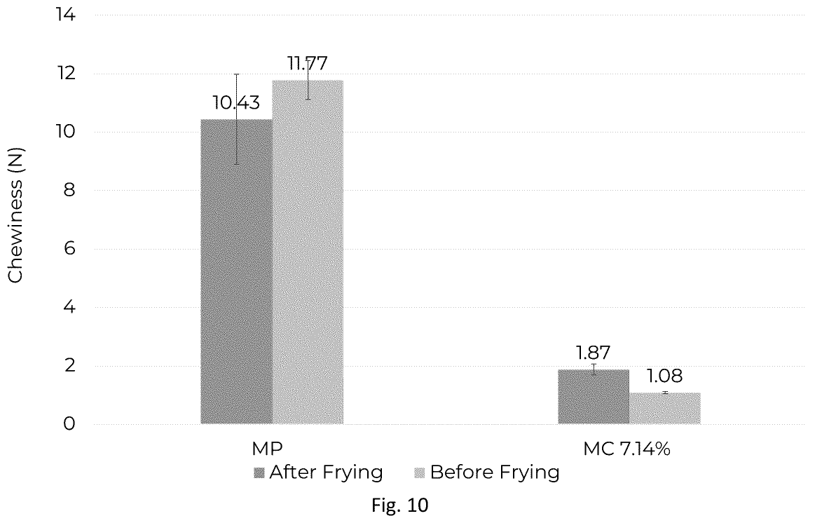

FIG. 10 is a graph comparing the gel results analysis for chewiness in accordance with some embodiments. Chewiness is defined as the energy required to masticate a solid food to a state ready for swallowing: a product of hardness, cohesiveness and springiness. In sensory terms, chewiness is a parameter used for solid foods and is a measure of how much energy is required to chew a particular foodstuff before it can be swallowed and is also a useful indicator for mouthfeel. In a TPA curve, this should be reported for solids and is defined as the product of gumminess*springiness (which equals hardness×cohesiveness×springiness):

$$Gumminess * ( Distance 2 / Distance 1 ) = Hardness * Cohesiveness * Springiness$$

### EXAMPLES

### Example 1—Preparation of Hydrogel

Several exemplary hydrogel formulations were prepared using soy protein, chickpea protein, pea protein and canola proteins. Numerous modifications, changes, variations, substitutions and equivalents will be apparent to those skilled in the art without departing from the spirit and scope of the exemplary hydrogel formulations, which follow.

The hydrogel was prepared by mixing protein with water. The protein water mixture was either heated and cooled or left untreated before adding the enzyme mixture in a ratio of about 1:0.01-0.05 protein to enzyme ratio, optionally along with a co-factor. If required, water was added during mixing to obtain a hydrogel with a desired consistency.

Non-limiting examples of formulas include:

Formula 1: The hydrogel was prepared as essentially set forth above by mixing isolated soy protein with water and adding an enzyme mixture containing transglutaminase and pectinmethylesterase with a protein to enzyme ratio of about 1:0.03.

Formula 2: The hydrogel was prepared as essentially set forth above by mixing isolated soy protein with water and adding laccase and pectinmethylesterase with a protein to enzyme ratio of 1:0.03.

Formula 3: The hydrogel was prepared as essentially set forth above by mixing isolated soy protein with water and adding transglutaminase and amylase at protein to enzyme ratio of 1:0.03.

Formula 4: The hydrogel was prepared as essentially set forth above by mixing isolated soy protein and an enzyme mixture containing laccase and amylase with a protein to enzyme ratio of 1:0.03 at the indicated ratio.

Formula 5: The hydrogel was prepared as essentially set forth above by mixing isolated pea protein with water and adding an enzyme mixture containing laccase and amylase with a protein to enzyme ratio of 1:0.04

Formula 6: The hydrogel was prepared as essentially set forth above by mixing isolated pea protein with water and adding an enzyme mixture containing transglutaminase and amylase with a protein to enzyme ratio of 1:0.04

Formula 7: The hydrogel was prepared as essentially set forth above by mixing isolated pea protein with water and an enzyme mixture containing transglutaminase and pectinmethylesterase with a protein to enzyme ratio of 1:0.04.

Formula 8: The hydrogel was prepared as essentially set forth above by mixing isolated pea protein and water and adding an enzyme mixture containing laccase and Pectinmethylesterase with a protein to enzyme ratio of 1:0.04.

Formula 9: The hydrogel was prepared as essentially set forth above by mixing isolated canola protein with water and adding an enzyme mixture including transglutaminase and amylase with a protein to enzyme ratio of 1:0.02.

Formula 10: The hydrogel was prepared as essentially set forth above by mixing isolated canola protein with water and adding enzyme mixture including laccase and amylase with a protein to enzyme ratio of 1:0.02.

Formula 11: The hydrogel was prepared as essentially set forth above by mixing isolated canola protein with water and adding enzyme mixture including transglutaminase and pectinmethylesterase with a protein to enzyme ratio of 1:0.02, respectively.

Formula 12: The hydrogel was prepared as essentially set forth above by mixing isolated canola protein with water and adding and enzyme mixture including laccase and Pectinmethylesterase with a protein to enzyme ratio of 1:0.02.

Formula 13: The hydrogel was prepared as essentially set forth above by mixing isolated canola protein with water and adding an enzyme mixture with a protein to enzyme ratio of 1:0.02.

Formula 14: The hydrogel was prepared as essentially set forth above by mixing isolated canola protein with water and adding an enzyme mixture including transglutaminase and amylase with a protein to enzyme ratio of 1:0.02.

Formula 15: The hydrogel is prepared as essentially set forth above by mixing isolated chickpea protein with water and adding an enzyme mixture including tyrosinase and amylase with a protein to enzyme ratio of 1:0.05.

Formula 16: The hydrogel is prepared as essentially set forth above by mixing isolated chickpea protein with water and adding an enzyme mixture including tyrosinase and pectinmethyltransferase with a protein to enzyme ratio of 1:0.05.

Formula 17: The hydrogel is prepared as essentially set forth above by mixing isolated chickpea protein with water and adding an enzyme mixture including tyrosinase and amylase with a protein to enzyme ratio of 1:0.03.

Formula 18: The hydrogel is prepared as essentially set forth above by mixing isolated chickpea protein with water and an enzyme mixture including tyrosinase and with a ratio of 1:0.05 along with vitamin C as cofactor.

Formula 19: The hydrogel is prepared as essentially set forth above by mixing isolated chickpea protein with water and adding an enzyme mixture including laccase and amylase with a protein to enzyme ratio of 1:0.05 and copper as a co-factor.

Formula 20: The hydrogel is prepared as essentially set forth above by mixing isolated chickpea protein with water and adding an enzyme mixture including laccase and cellulase with a protein to enzyme ratio of 1:0.03 and copper as a co-factor.

Formula 21: The hydrogel is prepared as essentially set forth above by mixing isolated sunflower protein with water and adding an enzyme mixture including laccase and cellulase at a protein to enzyme ratio of 1:0.03 and iron as a co-factor

Formula 22: The hydrogel is prepared as essentially set forth above by mixing isolated pea protein with water and adding an enzyme mixture including tranglutaminase and cellulase with a protein to enzyme ratio of 1:0.03 and calcium as a co-factor.

Formula 23: The hydrogel is prepared as essentially set forth above by mixing isolated sunflower protein with water and adding an enzyme mixture including laccase and cellulase at a protein to enzyme ratio of 1:0.05 and iron as a co-factor

Formula 24: The hydrogel is prepared as essentially set forth above by mixing isolated chickpea protein with water and adding an enzyme mixture including laccase and cellulase with a protein to enzyme ratio of 1:0.03.

Formula 25: The hydrogel is prepared as essentially set forth above by mixing a soy protein concentrate with an enzyme mixture including transglutaminase and pectinmethylesterase.

Formula 26: The hydrogel is prepared as essentially set forth above by mixing a soy protein concentrate with an enzyme mixture including transglutaminase, laccase and pectinmethylesterase.

Formula 27: The hydrogel is prepared as essentially set forth above by mixing isolated sunflower seed protein with an enzyme mixture including transglutaminase and pectinmethylesterase.

Additional formulations including other proteins derived from other plants whether in the form of a concentrate or isolated proteins are also prepared. Optionally, the hydrogel may be dehydrated to form a powder.

### Example 2—Water Retention and Cooking Loss

### 1. Methods

Three methods, which are listed below, were tested:
1. Cooking loss—by weighting the samples before baking and after baking.
   The baking procedure includes heating the samples at 100-110° C. for 15-20 min.

$$\% Cooking loss = ( 1 - \frac{Weight after baking}{Weight before baking} ) * 100$$

2. Cooking loss—by weighting the samples before frying and after frying at equal oil amount

$$\% Cooking loss = ( 1 - \frac{Weight after frying}{Weight before frying} ) * 100$$

3. Water holding Capacity—By centrifuging the samples coated with bakery paper or filter paper. Two groups of samples were tested, one group was centrifuge after frying while the second group was centrifuge without the frying before.

$$\% water holding capacity = ( \frac{Weight after centrifuge}{Weight before centrifuge} ) * 100$$

### 2. Results

The results are shown in Table 3 below, and demonstrate the high water capacity of the hereindisclosed hydrogel.

TABLE 3
water retention and cooking loss

| Sample I.D | Test Method | Cooking Loss | Water capacity |
|---|---|---|---|
| Plant (soy) based | Baking for 18 min at | 20.08% | |
| protein matrix: | 100° C. | 20.47% | |
| Transglutaminase | Baking for 15 min at | 19.98% | |
| (TG) + | 105º C. | | |
| Pectinmethylesterase | Frying | 11.33% | |
| (Formulation 1) | | 16.94% | |
| | Centrifuge without frying | | 88.21% |
| | before | | |
| | Centrifuge after frying | | 81.65% |
| Plant (soy) based | Frying | 20% | |
| protein matrix: | | 26% | |
| Laccase + | | | |
| Pectinmethylesterase | Centrifuge without frying | | 71.27% |
| (Formulation 2) | before | | |
| Methyl Cellulose | Baking for 18 min at | 19.84% | |
| | 100° C. | | |
| | Baking for 15 min at | 17.7% | |
| | 105º C | | |
| | Centrifuge after frying | | 83.33% |
| | Centrifuge without frying | | 89.10% |
| | before | | |

Similar results were also obtained for matrices including proteins from other sources (chickpea, pea and canola as well as for matrices utilizing amylase instead of pectinmethylesterase (formulations 5-15) data not shown).

The water retention and cooking loss of additional matrices, such as matrices including proteins obtained from other plant protein sources, using other enzymes is also evaluated.

### Example 3 Texture Analysis

In FIGS. 6-10, a TPA test was undertaken on a sample of the plant-based matrix (MP) (formulation 1) was compared with a sample of a methylcellulose matrix (MC), and a variety of parameters calculated therefrom. The TPA test was a double compression cycle performed at 10 mm/s until a recorded deformation of 50% was achieved, 2-4 repeats of each sample were performed. The sample size was about 33 mm in diameter and 2 cm height. Similar results were obtained from formulations 2-4 (not shown).

The TPA test is also carried out for additional formulations, such as but not limited to formulations 5-21.

The following parameters were used:
Test Mode—TPA
Pre-load Speed—20 mm/sec
Pre-load 0.1 N
Test Speed—10 mm/sec

FIG. 6 is a graph comparing the gel results analysis for hardness (N), defined as the highest peak force measured during first compression, in accordance with some embodiments. Hardness is the physical force necessary to attain a given deformation.

As seen from the figure, the herein disclosed plant-based gel (MP) advantageously has similar hardness before and after frying, whereas the methylcellulose gel (MC) shows greatly increased hardness after frying. This is an indication of the thermos-resistance of the herein disclosed hydrogels and is advantageous because a change in hardness as the food product cools down is unpleasant in the mouth, and may change the appearance and consistency of the food product.

FIG. 7 is a graph comparing the gel results analysis for cohesiveness in accordance with some embodiments. Cohesiveness is defined as the extent to which a material can be deformed before it ruptures.

The herein disclosed plant-based gel (MP) advantageously has similar cohesiveness before and after frying, whereas the methylcellulose gel (MC) shows greatly reduced cohesiveness after frying. A stable cohesiveness is essential because it is important that the food product not lose its consistency (e.g., fall apart) on cooking.

FIG. 8 is a graph comparing the gel results analysis for springiness in accordance with some embodiments. Springiness is the rate at which a deformed material returns to its undeformed condition after the deforming force is removed.

Both the herein disclosed plant-based gel (MP) and the methylcellulose gel (MC) show similar springiness before and after frying, however, advantageously the springiness of the MP is greater than that of the MC both before and after frying. Improved springiness is important as it is similar to the springiness found in animal proteins.

FIG. 9 is a graph comparing the gel results analysis for gumminess in accordance with some embodiments.

Both the herein disclosed plant-based gel (MP) and the methylcellulose gel (MC) show similar gumminess before and after frying, however, advantageously the gumminess of the MP is far greater than that of the MC both before and after frying. Improved gumminess is important as it is similar to the springiness found in animal proteins.

FIG. 10 is a graph comparing the gel results analysis for chewiness in accordance with some embodiments. Chewiness is defined as the energy required to masticate a solid food to a state ready for swallowing: a product of hardness, cohesiveness and springiness.

The chewiness of the herein disclosed plant-based gel (MP) is significantly higher than the chewiness of the methylcellulose gel (MC). This implies that the MP hydrogel advantageously feels less ‘squidgy’ during mastication, and has more structure compared to the MC gel.

### Example 4—Comparison to Albumen

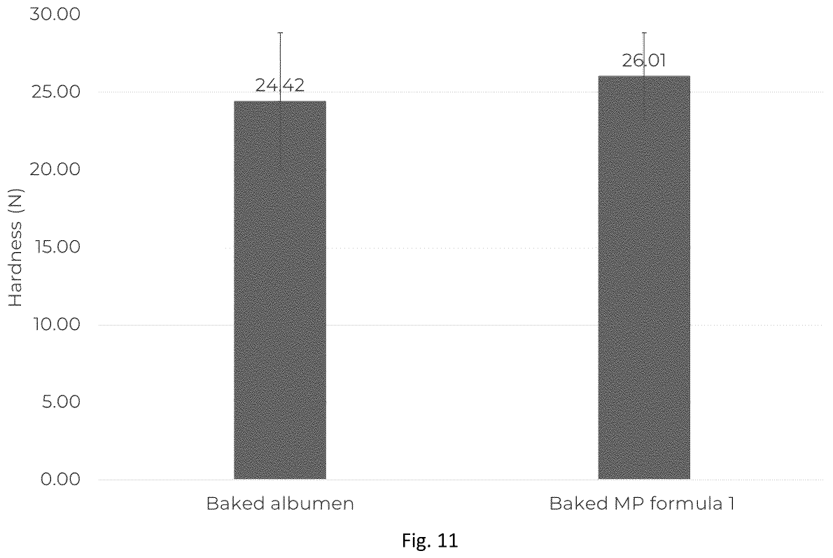

In FIGS. 11-14, show TPA test obtained for a sample of the hereindisclosed plant-based matrix (MP) (formulation 1) and for the egg white protein albumen. The TPA test was a double compression cycle performed at 10 mm/s until a recorded deformation of 50% was achieved, 2-4 repeats of each sample were performed. The sample size was about 33 mm in diameter and 2 cm height. Similar results were obtained from formulations 2-4 (not shown).

FIG. 11 is a graph comparing the gel results analysis for hardness (N), defined as the highest peak force measured during first compression, in accordance with some embodiments. Hardness is the physical force necessary to attain a given deformation.

As seen from the figure, the herein disclosed plant-based gel (MP) advantageously has similar hardness to that of the egg white protein (albumen) when baked for 40 min at 140° C., emphasizing the ability of the protein to serve as an egg white substitute.

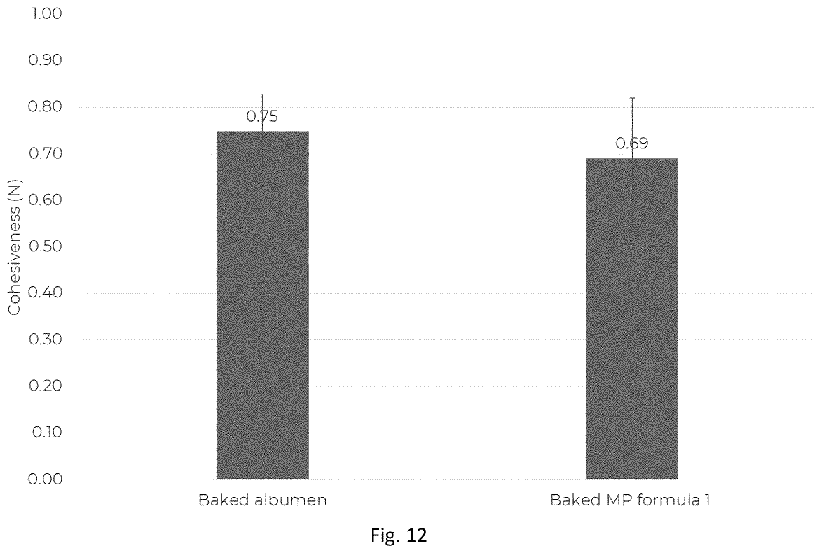

FIG. 12 is a graph comparing the gel results analysis for cohesiveness in accordance with some embodiments. Cohesiveness is defined as the extent to which a material can be deformed before it ruptures.

Again, the herein disclosed plant-based gel (MP) advantageously has similar cohesiveness to that of the egg white protein (albumen) when baked for 40 min at 140° C., emphasizing the ability of the protein to serve as an egg white substitute.

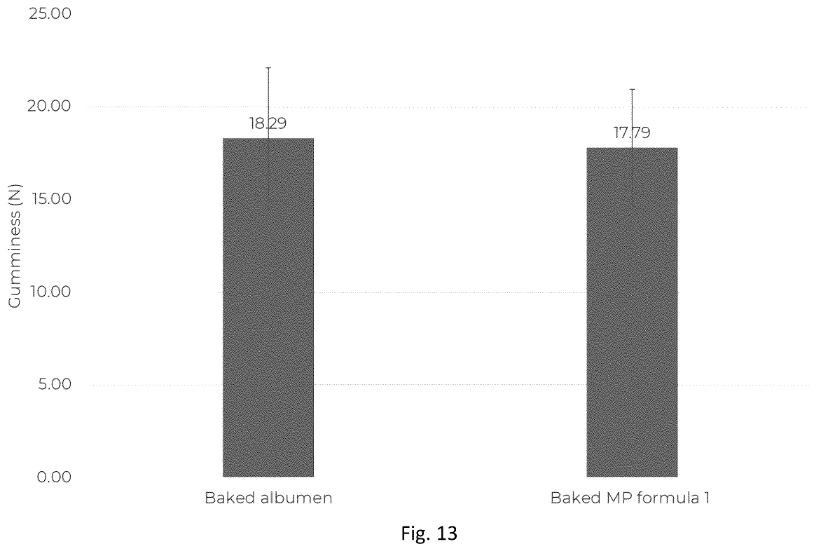

FIG. 13 is a graph comparing the gel results analysis for gumminess in accordance with some embodiments.

Again, as seen from the figure, the herein disclosed plant-based gel (MP) advantageously has similar gumminess to that of the egg white protein (albumen) when baked for 40 min at 140° C., emphasizing the ability of the protein to serve as an egg white substitute.

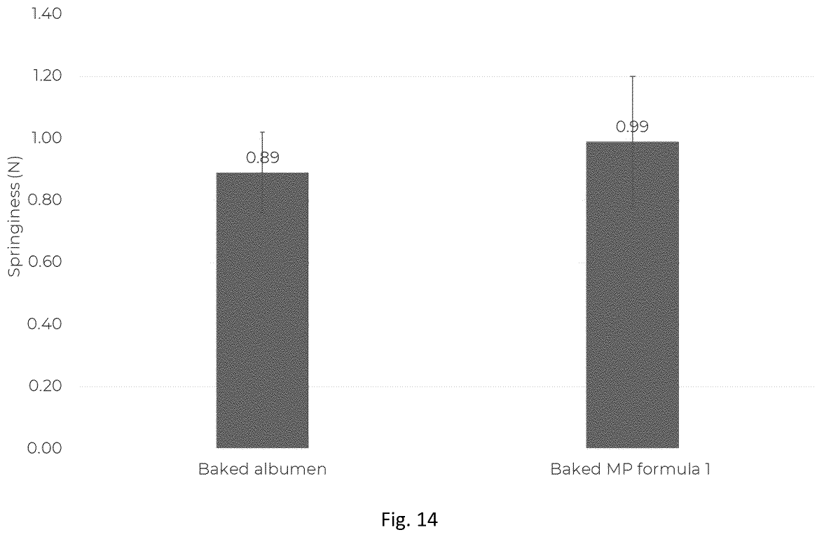

FIG. 14 is a graph comparing the gel results analysis for springiness in accordance with some embodiments. Springiness is the rate at which a deformed material returns to its undeformed condition after the deforming force is removed.

As seen from the graph, the herein disclosed plant-based gel (MP) advantageously has also a similar gumminess to that of the egg white protein (albumen) when baked for 40 min at 140° C., emphasizing the ability of the protein to serve as an egg white substitute.

FIG. 15 is a graph comparing the gel results analysis for chewiness in accordance with some embodiments. Chewiness is defined as the energy required to masticate a solid food to a state ready for swallowing: a product of hardness, cohesiveness and springiness.

As seen from the graph, the herein disclosed plant-based gel (MP) advantageously has also a similar chewiness to that of the egg white protein (albumen) when baked for 40 min at 140° C., emphasizing the ability of the protein to serve as an egg white substitute.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

In the description and claims of the application, the words “include” and “have”, and forms thereof, are not limited to members in a list with which the words may be associated.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “significant” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

Any methods and materials similar or equivalent to those described in WO2021138482A1 (WO'482), the teachings of which are incorporated herein by reference, can be used in the practice or testing of the methods, systems, and compositions described herein.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

1-38. (canceled)

39. A composition comprising a porous plant protein matrix comprising crosslinked plant derived polypeptides, a polysaccharide degrading enzyme, and at least one peptide bond forming enzyme and/or an oxidoreductase, wherein the matrix forms a hydrogel when hydrated, wherein the composition is essentially devoid of animal derived proteins and/or fats.

40. The composition of claim 39, being devoid of methyl cellulose.

41. The composition of claim 39, comprising at least 90% plant protein source, when in powder form.

42. The composition of claim 39, wherein the at least one enzyme capable of forming the peptide bonds is a transferase.

43. The composition of claim 42, wherein the transferase is an amino-acyltransferases.

44. The composition of claim 39, wherein the oxidoreductase is a multicopper enzyme capable of oxidating phenolic residues.

45. The composition of claim 39, wherein the polysaccharide bond degrading enzyme is a pectinase, pectinmethylesterase, an amylase, a cellulase or any combination thereof.

46. The composition of claim 39, being in the form of a powder.

47. The composition of claim 39, wherein the at least one polysaccharide degrading enzyme and/or the at least one peptide bond forming enzyme is reversibly inactivated by drying and/or freezing and wherein the reversibly inactive enzyme is reactivated upon hydration and/or thawing.

48. The composition of claim 39, wherein plant-based polypeptide is derived from the group consisting of pea, corn, wheat, rice, nuts, almond, peanut, seitan, lentil, chickpea, flaxseed, chia seed, oat, buckwheat, bulgur, millet, sunflower, canola, legumes, pulses, tofu, soy, tempeh, seitan, seeds, grain, chickpeas, lentils, legume, lupin, rapeseed, yeast, algae, microalgae, edamame, spelt, teff, hemp seeds, spirulina, amaranth, quinoa, leafy vegetables, oats, wild rice, chia seeds, fava bean, yellow pea, mung bean, nuts, protein-rich fruits and vegetables (such as broccoli, spinach, asparagus, artichokes, potatoes, sweet potatoes, brussels sprouts, sweet corn, guava, cherimoyas, mulberries, blackberries, nectarines, bananas).and combinations thereof.

49. The composition of claim 39, wherein the hydrogel is thermoresistant.

50. A food product comprising the composition of claim 39.

51. The food product of claim 50, being a plant-based meat alternative product, plant-based fish alternative product, egg-less egg alternative product, a dairy replacement product, a chocolate alternative product, an egg-less bakery product.

52. The food product of claim 50, wherein a change in a cohesiveness before and after cooking of the food product is at least 10% less than a change in the hardness obtained for the same food product including methylcellulose.

53. The food product of claim 50, wherein a change in a hardness before and after cooking of the food product is at least 10% less than a change in the hardness obtained for the same food product including methylcellulose.

54. The food product of claim 50, wherein a change in a springiness before and after cooking of the food product is at least 10% less than a change in the hardness obtained for the same food product including methylcellulose.

55. The food product of claim 50, wherein a change in a chewiness before and after cooking of the food product changes by less than 10% before and after cooking.

56. A process for producing a porous plant protein matrix capable of forming a hydrogel when hydrated, the process comprising:
a. mixing plant-derived polypeptides with at least one polysaccharide degrading enzyme and at least one peptide bond forming enzyme;
b. incubating at conditions allowing crosslinking of at least a portion of the plant derived polypeptides, thereby forming a hydrogel.

57. The process of claim 56, further comprising a step of preprocessing the plant-based polypeptides prior to and/or during the mixing to expose amino acid residues, wherein the preprocessing comprises, heating, pressure, sonication, extrusion, cold plasma, ultrasound, ultraviolet or any combination thereof.
